(19) United States
(12) Patent Application Publication
(10) Pub. No.: US 2009/0197298 A1
(43) Pub. Date: Aug. 6, 2009

(54) METHOD FOR DETECTING MICROORGANISMS IN A BIOLOGICAL SAMPLE

(76) Inventor: Alain RAMBACH, Paris (FR)

Correspondence Address: Baker Donelson Bearman, Caldwell & Berkowitz, PC, 555 Eleventh Street, NW, Sixth Floor, Washington, DC, 20004, US

(21) Appl. No.: 12/361,851

(22) Filed: Jan. 29, 2009

(30) Foreign Application Priority Data
Jan. 30, 2008 (FR) 08/50573

(51) Int. Cl. C12Q 1/04; C12M 1/34

(52) U.S. Cl. 435/34; 435/288.7

(57) ABSTRACT

This invention relates to a method for detecting at least one specific microorganism in a biological sample, said method comprising the following steps: a) culturing said biological sample in a culture medium, b) subjecting said culture medium to at least two radiations each presenting a specific wavelength, c) obtaining at least two different images of said culture medium using at least two reading systems, d) combining said at least two different images to produce at least one combined artificial image, and e) analyzing said combined artificial image to detect the presence of said at least one specific microorganism in the biological sample. The invention also relates to a device for application of the detection method according to this invention.

The present patent application claims the priority of the French patent application FR 08/50573 filed on Jan. 30, 2008, which is herein incorporated by reference.

### FIELD OF THE INVENTION

The present invention relates to a method for detecting the presence of specific microorganisms in a biological sample as well as a device allowing application of the detection method according to this invention.

### BACKGROUND

Rapid detection of microorganisms present in biological or food samples or samples from a hospital environment is essential in order to quickly establish the correct measures to be taken in the event of contamination.

It is important to be able to identify the contaminants without error in order to be able to take the appropriate clinical or industrial measures.

A rapid and effective system currently being developed to read Petri dishes is the use of readers in place of observation with the naked eye.

Current systems thus use a single light in the visible wavelength range and observations are carried out using reflected or transmitted light, or a single light in the UV range with observation carried out using a re-emitted light image. Reading is then generally carried out using a fixed reading system reading a surface (camera, etc.) or mobile system reading along the length of a wavelength (scanner, etc.). These systems provide a single image.

However these devices do not always provide sufficient and appropriate detection and this can give rise to errors in the identification of certain organisms.

### SUMMARY OF THE INVENTION

The present invention relates to a method for detecting at least one specific microorganism in a biological sample, said method comprising the following steps:
a) culturing said biological sample in a culture medium,
b) subjecting said culture medium to at least two radiations each presenting a specific wavelength,
c) obtaining at least two different images of said culture medium using at least two reading systems,
d) combining said at least two different images to produce at least one combined artificial image, and
e) analyzing said combined artificial image to detect the presence of said at least one specific microorganism in the biological sample.

The present invention further relates to a device for application of said method comprising:
at least two lighting systems, each lighting system emitting radiation of a specific wavelength,
at least two reading systems to obtain at least two images of the culture medium as defined for previously, and
at least one system for combining images obtained by the at least two reading systems in order to obtain a combined artificial image

### DESCRIPTION OF THE FIGURES

FIG. 1 is a diagrammatic representation of images of a culture medium according to the invention. The images 1 and 2 correspond to images obtained in step c), and the image 3 corresponds to a combined artificial image of images 1 and 2.

FIG. 2 is a diagrammatic representation of a detection device according to this invention.

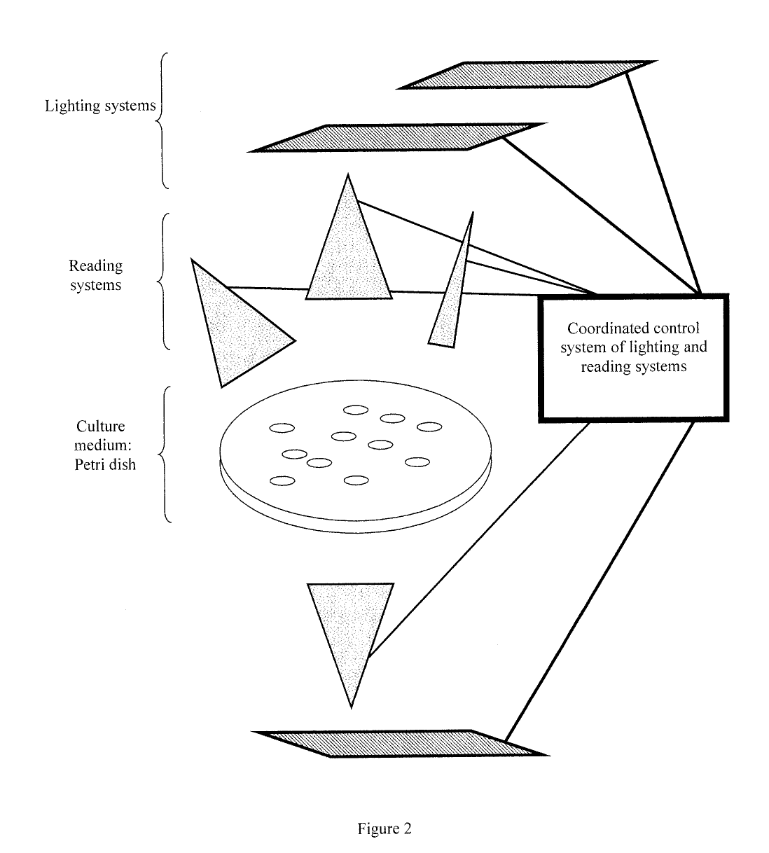

### DETAILED DESCRIPTION OF THE INVENTION

The invention proposes a solution to refine the results obtained by methods of the prior art by means of a method for detecting at least one specific microorganism in a biological sample, said method comprising the following steps:
a) culturing said biological sample in a culture medium,
b) subjecting said culture medium to at least two radiations each presenting a specific wavelength,
c) obtaining at least two different images of said culture medium using at least two reading systems,
d) combining said at least two different images to produce at least one combined artificial image, and
e) analyzing said combined artificial image to detect the presence of said at least one specific microorganism in the biological sample.

The term “microorganism” for example refers to bacteria or yeasts, preferably bacteria.

The term “specific microorganism” refers equally to a particular genus or species or group of microorganisms known to have common properties, such as resistance to an antimicrobial agent and currently identified by a common term.

As an example of specific microorganisms, we can cite bacteria or yeasts belonging to a genus (e.g. Staphylococcus) or specific species (e.g. Staphylococcus aureus) or bacteria or yeasts resistant to a particular antibiotic, said bacteria or yeasts also possibly belonging to a genus or defined species (e.g. MRSA).

Advantageously, the term “specific microorganism” refers to a particular genus or species and in a particularly preferred manner, a particular species.

The term “biological sample” refers to any type of microbiological sample, such as a food sample (dairy products, meat, etc.), soil sample, sample from a mammal (skin, mucous membranes, etc.) or one of their derivatives such as a preculture originating from such a sample.

Advantageously, said biological sample is a liquid biological sample such as saliva, blood or urine, a solid biological sample such as faeces, or a derivative of a liquid or solid biological sample such as a preculture from such a liquid or solid biological sample.

Yet more advantageously, said biological sample is a mixture of various microorganisms which can belong to distinct species or even genus. As an example, said biological sample corresponds to a mixture of at least two different microorganisms, preferably at least five different microorganisms and, in a particularly preferred manner, at least ten different microorganisms.

The term “culture medium” refers to a medium which allows growth of said at least one specific microorganism to be detected.

Said culture medium includes the necessary nutrients for growth of said at least one specific microorganism to be detected.

The term “nutrients needed for growth of said at least one specific microorganism to be detected” refers to the composition of a basic medium needed for said growth. The man skilled in the art is fully aware of the composition of said media and is able to adapt them if need be as a function of the specificity of certain microorganisms or restrictions linked to certain cases of this invention (for example, transparency of the medium). These nutrients are notably chosen from among the group consisting of carbon, nitrogen, sulphur, phosphorus, vitamins, growth factors, carbohydrates, salts (for example calcium, magnesium, manganese, sodium, potassium), nutritional complexes (for example amino acids, blood, serum, albumin) as well as peptones and animal tissue or vegetable extracts.

Preferably, said culture medium is a gelose medium, such a culture medium being, for example, agar-based. Utilisable culture media include Petri dishes which support the growth of microorganisms.

The culture medium according to this invention can also contain one or more antimicrobial agents, in particular one or more antibiotics and/or one or more antifungal agents.

Said antimicrobial agent(s) makes it possible to restrict the growth of microorganisms other than said at least one specific microorganism to be detected.

The efficacious amount of antimicrobial agent to be used can be established quite simply by the man skilled in the art on the basis of his general knowledge.

The term “culturing” refers to inoculation of said culture medium with all or some of the biological samples and incubation of said inoculated culture medium.

The man skilled in the art can adapt the incubation conditions as a function of the culture medium, biological sample and specific microorganism to be detected using his general knowledge.

The incubation step can be carried out at a temperature of around 30° C. to 40° C., preferably 37° C., for a duration of about 18 to 24 h. Nevertheless, depending on the means available, the man skilled in the art can adapt the temperature and duration of this incubation step on the basis of his general knowledge.

The term “lighting system” is a system allowing the culture medium to be subjected to radiation of a specific wavelength.

The lighting system can emit radiations in the visible or non-visible range. For the purposes of the invention, it should be understood that “lighting system” refers to a system which emits radiations of a particular wavelength or a particular range of wavelengths.

For the purposes of this invention, said lighting system can consist uniquely of ambient light, for example daylight.

Advantageously, the method according to the invention therefore comprises the following steps:
a) culturing of said biological sample in a culture medium, said culture medium including at least one reporter compound,
b) subjecting said culture medium to radiations of at least two different wavelengths, at least one of said wavelengths corresponding to the absorption wavelength of at least one reporter product or product released by the reporter compound present in said culture medium,
c) obtaining at least two different images of said culture medium using at least two reading systems, at least one of the two reading systems allowing detection of at least one reporter compound or product released by the reporter compound,
d) combining said images obtained in step c) to produce at least one combined artificial image,
e) analyzing the combined artificial image obtained in step d) to detect the presence of said at least one specific microorganism in the biological sample.

The term “reporter compound” refers to a dye material, chromogenic agent or fluorogenic agent allowing detection of said at least one specific microorganism.

Examples of dyes include neutral red present in “MacConkey Agar” medium or else the green dye and the black precipitating dye linked to the H2S characteristic present in “Hektoen” medium.

Preferably, the reporter compounds present in said sample are chosen from among the group consisting of chromogenic agents and fluorogenic agents.

The term “chromogenic agent” or “fluorogenic agent” refers to an enzyme substrate capable of releasing respectively a chromophore or a fluorophore under the effect of this enzyme, said chromophore or fluorophore becoming detectable when it is subjected to an appropriate wavelength.

The term “product released by a reporter compound” refers to a chromophore or fluorophore in the case of a chromogenic agent or fluorogenic agent respectively.

Said chromogenic agent or fluorogenic agent is the substrate of an enzyme expressed by the specific microorganism to be detected or an enzyme expressed by another microorganism and also likely to be present in the biological sample. For example, in the presence of a bacterial species to be detected expressing an enzyme allowing hydrolysis of a chromogenic agent, the chromophore is released and stains said specific microorganism a given colour.

Preferably, said at least one reporter compound is present in said culture medium prior to inoculation with the biological sample. Alternatively, said at least one reporter compound can be added at the same time or after inoculation of said culture medium before the biological sample or it may be present in the biological sample prior to inoculation of said culture medium with said biological sample.

Advantageously, the culture medium of the method of this invention can include a combination of several reporter compounds such as for example 2, 3, 4, 5, 6 or more reporter compounds.

Advantageously again, the method according to the invention comprises the following steps:
a) culturing said biological sample in a culture medium, said culture medium including at least two reporter compounds,
b) subjecting said culture medium to radiation of at least two different wavelengths, each of said wavelengths corresponding to the absorption wavelength of one of the at least two reporter compounds or products released by reporter compounds present in said culture medium,

c) obtaining at least two different images of said culture medium using at least two reading systems, each of the two reading systems allowing detection of at least one of the two reporter compounds or products released by the reporter compounds,
d) combining said images obtained in step c) into at least one combined artificial image,
e) analysing the combined artificial image obtained in step d) to detect the presence of said at least one specific microorganism in the biological sample.

Evidently, the combination of said reporter compounds is established as a function of the different specific microorganisms that are to be detected, and more particularly as a function of the respective enzyme activities of said microorganisms.

Preferably, the culture medium according to this invention includes at least one chromogenic agent and at least one fluorogenic agent, said combination being chosen to allow detection of said at least one specific microorganism in the biological sample.

Within the scope of this invention, it is necessary to establish the combination of chromogenic agent(s) and fluorogenic agent(s) suited to detection of the specific microorganism to be detected.

The choice of combination of chromogenic agents and fluorogenic agents is of vital importance but it is in no way necessary that the enzymes which act on these agents should be expressed only in the specific microorganisms to be detected. Thus the negative character of certain enzymes and/or positive character of certain enzymes of the specific microorganism to be detected are used so that the final spectrum, in terms of absorption wavelengths, can be used to identify said specific microorganism as a function of the chromophore(s) released or fluorophore(s) released.

According to a preferred embodiment of the method of the invention, said at least one chromogenic agent used is chosen from among enzyme substrates involved in sugar metabolism and notably in the group consisting of β-glucosidase substrates, β-galactosidasesubstrates, β-glucuronisase substrates and phosphatase substrates.

Examples of chromogenic agents which can be used to implement the method according to this invention include compounds leading to the production of hydrogen sulphide (H2S) which result in black staining under visible light.

Preferably, the chromophore is chosen from the group consisting of derivatives of indoxyl, halogeno-indoxyl (bromo-indoxyl, chloro-indoxyl, fluoro-indoxyl, iodo-indoxyl, dichloro-indoxyl, chloro-bromo-indoxyl, tri hloro-indoxyl), methyl-indoxyl, and hydroxyquinoline, in particular from the following derivatives: 6 chloro-indoxyl, 5-bromo-indoxyl, 3-bromo-indoxyl, 6-fluoro-indoxyl, 5-iodo-indoxyl, 4,6-dichloro-indoxyl, 6,7-dichloro-indoxyl, 5-bromo-4-chloro-indoxyl, 5-bromo-6-chloro-indoxyl, 4,6,7-trichloro-indoxyl, N-methyl-indoxyl or 8-hydroxy-quinoline.

According to another preferred embodiment of the method of the invention, said at least one fluorogenic agent used is chosen from among substrates of enzymes involved in sugar metabolism and notably from the group consisting of β-glucosidase substrates, β-galactosidase substrates, β-glucuronisase substrates and phosphatase substrates.

Examples of fluorogenic agents include 4-methyl umbelliferyl, for example bound to a glucose (Methyl-umbellyferyl-β-glucoside), or derivatives of ELF® such as ELF®-phosphate (MOLECULAR PROBES INC and BIOSYNTH) which release a precipitating fluorophore such as that described in patent application EP0949266.

According to a fifth preferred embodiment of the method of the invention, the culture medium in step a) consists of a chromogenic agent leading to the release of H2S and a fluorogenic agent leading to the release of methyl-umbellyferyl-P-glucoside.

The released hydrogen sulphide is then detected in the visible range and methyl-umbellyferyl-β-glucoside is detected in the ultraviolet wavelength range.

This preferred embodiment allows detection of strains of Salmonella in a biological sample including for example Salmonella, Citrobacter freundli and Escherichia coli bacteria. Such an embodiment is illustrated in example 1.

Preferably, subjecting the culture medium to radiation of at least two different wavelengths is carried out using at least two lighting systems.

Preferably, the lighting system makes it possible to subject the culture medium to radiation in a specific wavelength range for detection of a reporter compound or a product released by a reporter compound.

Preferably, the lighting system is chosen such that it emits at an absorption wavelength of a reporter compound or product released by a reporter compound present in said culture medium.

In still another preferred embodiment of the method according to the invention, the number of lighting systems depends on the type of reporter compounds present in the culture medium (chromogenic agents and/or fluorogenic agents). Advantageously, the number of lighting systems is equal to the number of types of reporter compounds present in the culture medium.

The visible spectrum extends from 400 to 800 nm, the infrared spectrum from 780 nm to 1000 μm and the ultraviolet spectrum from 10 to 400 nm.

Said lighting system can also allow transmission of reflected or transmitted light.

Said lighting system can also emit radiation in a continuous or discontinuous mode, for example in the order of a tenth of a second. For example, several lighting systems can be used to alternate lighting in the visible and non-visible ranges and produce in some cases a single resulting image visible to the naked eye.

Preferably, said at least two radiations emitted by said lighting systems have very different wavelengths.

As an example, the detection method according to this invention can use a lighting system which emits in the visible range and a lighting system which emits in the ultraviolet range, these two systems functioning in an alternating manner.

For example, the lighting system can consist of light emitting diodes (LED).

The term “image” obtained by the reading system refers for example to an optical image or numerical image or even an image obtained on a paper or plastic support. Preferentially, this is a numerical image.

The term “reading system” is a system which makes it possible to obtain at least one image of the culture medium.

The reading system can be fixed and read a specific surface (for example a camera) or it can be mobile in which case reading is carried out by scanning a particular wavelength (for example a scanner).

Preferably, the reading system makes it possible to detect a reporter compound or product released by a reporter compound in said culture medium when this is subjected to radiation from the lighting system at an appropriate wavelength.

Preferably, the reading system makes it possible to detect an emission wavelength of the reporter compound or a compound released by a reporter compound, for example in the visible, ultraviolet or infrared wavelength range.

The man skilled in the art can easily establish an appropriate reading system for detection of a given wavelength.

Thus advantageously use of at least two reading systems makes it possible to obtain at least two simultaneous images which can then be combined.

In a particular embodiment of the invention, the reading system can include at least one filter. For the purposes of the invention, it should be understood that the reading system covered with a filter differs from the reading system without a filter. If images are obtained with and without filters, it should therefore be understood that the system for obtaining images from a sample consists of two reading systems.

Preferably, the number of reading systems of the invention depends on the number of types of reporter compounds present in the culture medium. Advantageously, the number of reading systems is equal to the number of types of reporter compound present in the culture medium.

Combining the images in order to obtain a combined artificial image such as that described in step d) can therefore be carried out using an image combination system.

The term “combined artificial image” refers to superimposition of at least two different images obtained in step c). Preferably, the combined artificial image according to this invention is a two-dimensional image.

The combined artificial image can for example be an optical image or a numerical image or an image obtained on a paper or plastic support. Preferably, this is a numerical image.

The term “image combination system” refers to a system which receives different images and is then capable of combining these images, for example by superimposition, in order to obtain at least one combined artificial image which can then be used as a support for later analysis.

Advantageously, said image combination system is an image combination software such as that known to the man skilled in the art or possibly manual superimposition of at least two images. Preferably, said image combination system consists of an image combination software.

Analysis of the combined artificial image as described in step e) can be carried out by simple observation by the man skilled in the art or processed by computer.

Preferably, analysis of the combined artificial image of step e) is carried out by computer processing.

Preferably, the detection method according to this invention is a direct detection method.

The term “direct detection method” refers to a method which does not include a preliminary isolation step of the different microorganisms present in the biological sample. If need be, said direct detection method can include an enriching step, for example in a selective broth constituting a step of preculturing of said biological sample.

Advantageously, combination in step d) is carried out immediately after or simultaneously to step c).

The detection method proposed by this invention is essentially a qualitative test, in other words a test which reveals the presence or absence of a specific microorganism in the biological sample. Nonetheless, there is nothing to prevent the method of the invention being applied to quantitative measurement by means of successive dilutions and spreading on gelose medium or modification into a semi-quantitative test in accordance with the MPN method (Most Probable Number) for example.

The method of the invention is advantageously applied in a semi-automated manner and possibly a fully automated manner.

Another aspect of the invention relates to a device for application of the method according to the invention comprising:
at least two lighting systems, each lighting system emitting radiation of a specific wavelength,
at least two reading systems to obtain at least two images of the culture medium,
at least one system for combining images obtained by the at least two reading systems in order to obtain a combined artificial image.

Advantageously, said device for application of the method according to the invention comprises:
at least two lighting systems, at least one of said lighting systems emitting radiation at a wavelength corresponding to the absorption wavelength of a reporter compound or compound released by a reporter compound present in the culture medium as described previously,
at least two reading systems for obtaining at least two images of the culture medium, at least one of said reading systems allowing detection of a reporter compound or product released by the reporter compound,
at least one system for combining images obtained by the at least two reading systems in order to obtain a combined artificial image.

Preferably, said device for application of the method according to the invention includes:
at least two lighting systems, each lighting system emitting radiation at a wavelength corresponding to the absorption wavelength of a reporter compound or a compound released by the reporter compound present in said culture medium,
at least two reading systems, each reading system allowing detection of a reporter compound or product released by the reporter compound to obtain at least two images of the culture medium.
at least one system for combining images obtained by the at least two reading systems in order to obtain a combined artificial image.

Said device can also include a culture medium for application of the method according to the invention, possibly comprising at least one or more reporter compounds, preferably two reporter compounds. Said culture medium and said reporter compounds are as defined earlier.

Finally, said device for application of the method according to the invention can be controlled by computer.

The examples and figures below are intended to illustrate the invention without limiting its scope in any way.

### EXAMPLES

### Example 1

### Detection of Salmonella, Citrobacter freundii and Escherichia coli Strains in a Mixture

A mixture containing Salmonella, Citrobacter freundii and Escherichia coli strains is cultured in growth medium allowing production of H2S (hydrogen sulphide) and methyl-umbellyferyl-β-glucoside.

The nature of H2S production allows positive bacteria to be stained black.

Methyl-umbellyferyl-β-glucoside allows observation of fluorescence for positive bacteria under UV light.

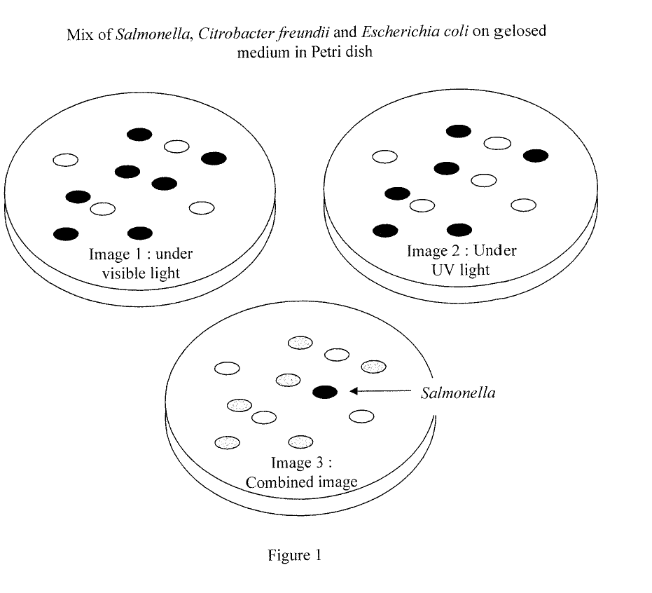

The results in table 1 below and FIG. 1 show that combining the image obtained under visible light and the image obtained under ultraviolet light allows a clear distinction to be made between the Salmonella strain which results in intense artificial Red staining and the Citrobacter freundii and Escherichia coli strains which result in artificial Pale Blue and Grey staining.

TABLE 1

| | Growth medium including the | | |
|---|---|---|---|
| | H2S producing characteristic (stained | | |
| | black for positive bacteria) and | | |
| | including Methyl umbellyferyl | | |
| | beta Glucoside (giving fluorescence | | |
| | for positive bacteria) | | |
| | | Citrobacter | |
| | Salmonella | freundii | E. coli |
| Blackening under visible | + | + | − |
| lighting | | | |
| Florescence under UV | − | + | − |
| lighting | | | |
| Combined artificial | Intense Red | Pale Blue | Grey |
| image (colours as a | | | |
| function of | | | |
| computer | | | |
| processing) | | | |

### Example 2

### Detection of Escherichia coli, Staphylococcus Enterococcus faecalis and Enterobacter cloacae in a Mixture

A mixture containing strains of Escherichia coli, Staphylococcus saprophyticus, Enterococcus faecalis and Enterobacter cloacae is cultured on CHROMagar Orientation growth medium (CHROMagar, Paris, France).

Colonies of these four species are stained red or blue under perpendicular lighting with white light.

The opacity of the colonies is read separately using processing of images obtained under angular lighting with white light.

The results given in table 2 below show that combining the image obtained under perpendicular lighting and the image obtained under angular lighting makes it possible to distinguish between the four strains using the four artificial colours obtained (Red, Grey, Yellow and Blue respectively).

TABLE 2

| | CHROMagar Orientation growth medium | | | |
|---|---|---|---|---|
| | (CHROMagar, Paris, France) stains bacterial | | | |
| | colonies differentially depending on the | | | |
| | species and giving an opaque appearance | | | |
| | to Gram-positive bacterial colonies | | | |
| | | Staphylococcus | Enterococcus | Enterobacter |
| | E. coli | saprophyticus | faecalis | cloacae |
| Red or blue | Red | Red | Blue | Blue |
| staining | | | | |
| Opaque | − | + | + | − |
| appearance | | | | |
| under angular | | | | |
| lighting | | | | |
| Combined | Red | Grey | Yellow | Blue |
| artificial | | | | |
| image | | | | |
| (colours as a | | | | |
| function of | | | | |
| computer | | | | |
| processing) | | | | |

We claim:

1. A method for detecting at least one specific microorganism in a biological sample, said method comprising the following steps:
a) culturing said biological sample in a culture medium,
b) subjecting said culture medium to at least two radiations each presenting a specific wavelength,
c) obtaining at least two different images of said culture medium using at least two reading systems,
d) combining said at least two different images to produce at least one combined artificial image, and
e) analyzing said combined artificial image to detect the presence of said at least one specific microorganism in the biological sample.

2. The method of claim 1, wherein said method comprises the following steps:
a) culturing of said biological sample in a culture medium, said culture medium including at least one reporter compound,
b) subjecting said culture medium to radiation of at least two different wavelengths, at least one of said wavelengths corresponding to the absorption wavelength of at least one reporter product or product released by the reporter compound present in said culture medium,
c) obtaining at least two different images of said culture medium using at least two reading systems, at least one of the two reading systems allowing detection of at least one reporter compound or product released by the reporter compound,
d) combining said images obtained in step c) to produce at least one combined artificial image, and
e) analyzing the combined artificial image obtained in step d) to detect the presence of said specific microorganism in the biological sample.

3. The method of claim 2, wherein said method comprises the following steps:
a) culturing said biological sample in a culture medium, said culture medium including at least two reporter compounds,
b) subjecting said culture medium to radiation of at least two different wavelengths, each of said wavelengths corresponding to the absorption wavelength of at least one reporter product or product released by the reporter compound present in said culture medium,
c) obtaining at least two different images of said culture medium using at least two reading systems, each of the two reading systems allowing detection of at least two reporter compounds or product released by the reporter compounds,
d) combining said images obtained in step c) to produce at least one combined artificial image, and
e) analyzing the combined artificial image obtained in step d) to detect the presence of said specific microorganism in the biological sample.

4. The method of claim 1, wherein said at least one specific microorganism to be detected is a bacterium.

5. The method of claim 2, wherein said at least one reporter compound present in said culture medium is chosen from the group comprising chromogenic agents and fluorogenic agents.

6. The method of claim 5, wherein said culture medium includes at least one chromogenic agent and at least one fluorogenic agent.

7. The method of claim 6, wherein said chromogenic agent leads to the release of H2S and said fluorogenic agent leads to the release of methyl-umbellyferyl-β-glucoside.

8. The method of claim 4, wherein said method is for the detection of bacteria of genus Salmonella.

9. The method of claim 1, wherein said culture medium also includes at least one antibiotic.

10. The method of claim 1, wherein said culture medium is a gelose medium.

11. A device for application of the method of claim 1 comprising:
at least two lighting systems, each lighting system emitting radiation of a specific wavelength,
at least two reading systems to obtain at least two images of the culture medium as defined in claim 1,
at least one system for combining images obtained by the at least two reading systems in order to obtain a combined artificial image.

12. The device of claim 11, wherein said device comprises:
at least two lighting systems, at least one of said lighting systems emitting radiation at a wavelength corresponding to the absorption wavelength of a reporter compound or compound released by a reporter compound present in said culture medium,
at least two reading systems for obtaining at least two images of the culture medium, at least one of said reading systems allowing detection of a reporter compound or product released by the reporter compound,
at least one system for combining images obtained by the at least two reading systems in order to obtain a combined artificial image.

13. The device of claim 11, wherein said device comprises:
at least two lighting systems, each lighting system emitting radiation at a wavelength corresponding to the absorption wavelength of a reporter compound or a compound released by the reporter compound present in said culture medium,
at least two reading systems, each reading system allowing detection of a reporter compound or product released by the reporter compound to obtain at least two images of the culture medium,
at least one system for combining images obtained by the at least two reading systems in order to obtain a combined artificial image.
